(19) United States
(12) Patent Application Publication
(10) Pub. No.: US 2023/0127849 A1
(43) Pub. Date: Apr. 27, 2023

(54) INTELLIGENT USER INTERFACE AND APPLICATION FOR LICENSE MANAGEMENT

(71) Applicant: CERNER INNOVATION, INC., Kansas City, KS (US)

(72) Inventors: Jyotsna Pamadi, Chennai (IN); Mani Kanta Ankam, Gudivada (IN); Mansoor Mohammed, Hyderabad (IN)

(21) Appl. No.: 17/507,066

(22) Filed: Oct. 21, 2021

(51) Int. Cl. G16H 40/20; G06F 40/177; G06F 16/248

(52) U.S. Cl. CPC G16H 40/20; G06F 40/177; G06F 16/248

(57) ABSTRACT

Methods, systems, and computer-readable media for intelligent license management are disclosed. A renewal date, complexity level, and contact information for a plurality of licenses is received and stored in a database, and a table comprising this information is generated and stored in the database. A reminder date for each of the plurality of licenses is determined based on the renewal date and complexity level and stored in the table in association with a corresponding license of the plurality of licenses. It is subsequently determined when a current date corresponds to the reminder date for a license, and a notification is automatically transmitted to a user associated with the contact information for the at least one license. The notification is configured to be displayed on a graphical user interface with a selectable link which when selected, causes display of the table.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description.

One aspect of the present disclosure relates to a method comprising receiving a renewal date, a complexity level, and contact information for each of a plurality of licenses in a database that is associated with a medical facility. At least the renewal date, the complexity level, and the contact information for each of the plurality of licenses is stored in the database, and a table of the renewal date, the complexity level, and the contact information for each of the plurality of licenses is stored in the database. A reminder date for each of the plurality of licenses is determined based on the renewal date and the complexity level that corresponds to each of the plurality of licenses, and the reminder date is stored in the table, wherein the reminder date is stored in association with a corresponding license of the plurality of licenses. The method also comprises subsequently determining when a current date corresponds to the reminder date for at least one license of the plurality of licenses. Based on determining that the current date corresponds to the reminder date for the at least one license, a notification is automatically transmitted to a user associated with the contact information for the at least one license, wherein the notification is configured to be displayed on a graphical user interface with a selectable link. Based on receiving an indication of a selection of the selectable link, at least a portion of the table is displayed on a user device associated with the user.

Another aspect of the present disclosure relates to a non-transitory computer readable media having computer-executable instructions embodied thereon that, when executed, perform a method. The non-transitory computer readable media comprises a computer application plug-in configured for receiving a renewal date, a complexity level, and contact information for each of a plurality of licenses in a database that is associated with a facility, wherein the complexity level defines a quantity of days that are needed to process a renewal of a corresponding license, wherein the renewal date defines a date for which the corresponding license is to expire, and wherein the contact information defines a user identifier and a contact method that defines a user to be contacted as responsible for management of the corresponding license. A table of the renewal date, the complexity level, and the contact information for each of the plurality of licenses, is generated and stored wherein the table is stored in the database that is associated with a particular facility, and wherein the plurality of licenses correspond to one or more licenses that govern personnel or equipment at the particular facility. A reminder date is automatically calculated for each of the plurality of licenses based on the renewal date and the complexity level that corresponds to each of the plurality of licenses, and for each day of a calendar year, it is automatically determined whether a current date corresponds to the reminder date for one or more of the plurality of licenses. For each of the one or more licenses in the plurality of licenses for which the current date corresponds to the reminder date, a notification is automatically transmitted to the user associated with the contact information for the one or more licenses, wherein the notification is configured to be displayed on a graphical user interface with a selectable link. Based on receiving an indication of a selection of the selectable link, at least a portion of the table is displayed on a user device associated with the user.

Yet another aspect of the present disclosure relates to a computer system comprising a memory, one or more processors, and one or more computer storage media storing computer-usable instructions that, when used by the one or more processors, cause the one or more processors to receive a renewal date, a complexity level, and contact information for each of a plurality of licenses in a database that is associated with a medical facility, and store at least the renewal date, the complexity level, and the contact information for each license of the plurality of licenses in the database. The system generates a table of the renewal date, the complexity level, and the contact information for each of the plurality of licenses, wherein the table is stored in the database. A reminder date for each of the plurality of licenses is determined based on the renewal date and the complexity level that corresponds to teach of the plurality of licenses. The reminder date for each of the plurality of licenses is stored in the table, wherein the reminder date is stored in association with a corresponding license of the plurality of licenses. It is subsequently determined when a current date corresponds to the reminder date for at least one license of the plurality of licenses. Based on determining that the current date corresponds to the reminder date for the at least one license, a notification is automatically transmitted to a user associated with the contact information for the at least one license, wherein the notification configured to be displayed on a graphical user interface of a user device with a selectable link. Based on receiving an indication of a selection of the selectable link, at least a portion of the table is displayed on the user device associated with the user.

### BRIEF DESCRIPTION OF THE DRAWINGS

Illustrative embodiments of the present disclosure are described in detail below with reference to the attached drawing figures, and wherein:

FIG. 1 illustrates an exemplary flow chart for uploading and managing licenses, in accordance with one or more implementations;

FIG. 2 illustrates an exemplary license management and notification system for generating notifications for renewal of licenses, in accordance with one or more implementations;

FIG. 3 illustrates an exemplary flow chart for intelligently generating notifications for license renewal, in accordance with one or more implementations;

FIG. 4 illustrates a flow chart of an example method for generating an intelligent user interface and application for license management, in accordance with one or more implementations;

FIG. 5 illustrates an example of a graphical user interface, in accordance with one or more implementations;

FIG. 6 illustrates an example of a license management notification for display in a graphical user interface, in accordance with one or more implementations;

FIG. 7 illustrates an exemplary flow chart for intelligently generating license management notifications for teams; and

FIG. 8 illustrates an example computing environment, in accordance with one or more implementations.

### DETAILED DESCRIPTION

The subject matter of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described. Further, it will be apparent from this Detailed Description that the technological solutions disclosed herein are only a portion of those provided by the present disclosure. As such, the technological problems, solutions, advances, and improvements expressly referenced and explained should not be construed in a way that would limit the benefits and application of embodiments of the present disclosure.

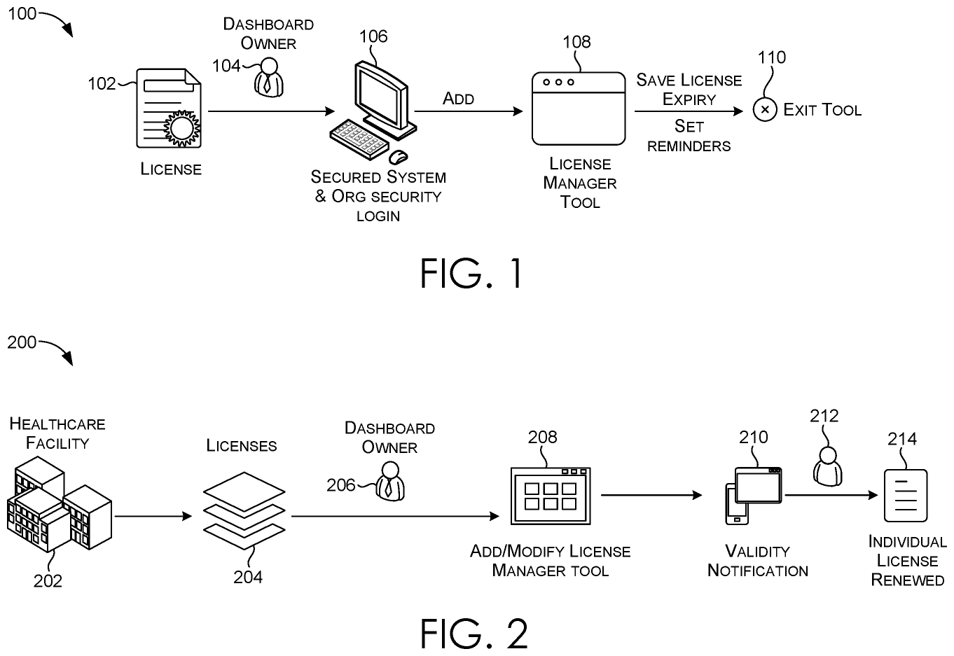

Aspects and embodiments herein provide an intelligent license management application having a graphical user interface “dashboard” that presents facility-wide or entity-wide tracking, renewal, and reporting functionalities for license involving equipment, building, personnel (staff, employees, contractors), software, or any combination thereof. The intelligent license management application provides a centralized cohort for management of all the licenses associated with an entity, including equipment, building, personnel, software, or any combination thereof. FIG. 1 illustrates an exemplary license input flow chart 100 in this regard. In embodiments, a license 102 may be manually input into the intelligent license management application by a dashboard owner 104. As used herein, a license may be any physical or virtual document or identifier that indicates or certifies the satisfactory presence of one or more regulated qualifications of equipment, building, personnel (staff, employees, contractors), software, or any combination thereof that are associated with any number of facilities, employers, entities, or any combination thereof. For example, a license associated with a staff member may be a medical license whereas a license associated with equipment may be an elevator maintenance certificate. In another example, a license associated with an associated company or associated employee may be a pilot's license certifying one or more persons as trained, eligible, and permitted to operate a medical helicopter for emergency transport of patients, clinicians, equipment, and/or biological material (e.g., donated organs) within a particular defined geographic region (e.g., United States of America). Examples are discussed throughout in the general context of a healthcare facility or entity for illustrative purposes only, but this is just one example and is not intended to be limiting in any manner because other types of facilities are contemplated and are within the scope of this disclosure.

The dashboard owner may be any individual having access to the intelligent license management application, displayed in a graphical user interface. This individual may be directly associated with a healthcare facility, or may be associated with a license management position for multiple healthcare facilities. In embodiments, the dashboard owner 104 may be associated with a defined privilege level that governs or electronically controls the individual's access to the intelligent license management application, the accessibility of one or more functionalities (e.g., read only access; read and write access) in the intelligent license management application, and/or the information that is displayed in a graphical user interface, for example, for security purposes.

In one example, a dashboard owner 104 may be associated with a standard privilege level that enables the individual to manually input only the license 102 to which they are associated. For example, a physician associated with a facility may only have the standard privilege level when acting as a dashboard owner 104 viewing the graphical user interface and interacting with the intelligent license management application. Therefore, the physician may use the intelligent license management application to input their own medical license for the state(s) in which they practice and/or a board certification, or any other license to which they are directly associated. In embodiments, these licenses could range from medical license to any number of licenses associated with particular procedures or medical equipment associated with a healthcare facility.

In another example, the dashboard owner 104 may be associated with an administrative privilege level that provides complete access to all functionalities and all license information within the intelligent license management application. In this embodiment, the dashboard owner 104 may be able to input any number and type of license irrespective of the individual's association or lack thereof with the licenses. For example, a manager acting as the dashboard owner 104 associated with a plurality of facilities may have the administrative privilege level. This manager may be able to input any number and of license associated with employees of a healthcare facility, such as physicians, nurses, pharmacists, nuclear medicine technologists, respiratory therapists, and the like. The manager having the administrative privilege level may also input licenses associated with facilities, such as structural building permits, elevator safety and operational inspections, medical transport vehicles inspections, and the like. For example, the manager may be able to input licenses associated with equipment such as different medical devices (e.g., imaging equipment, linear accelerators or cobalt machines for external beam therapy delivery).

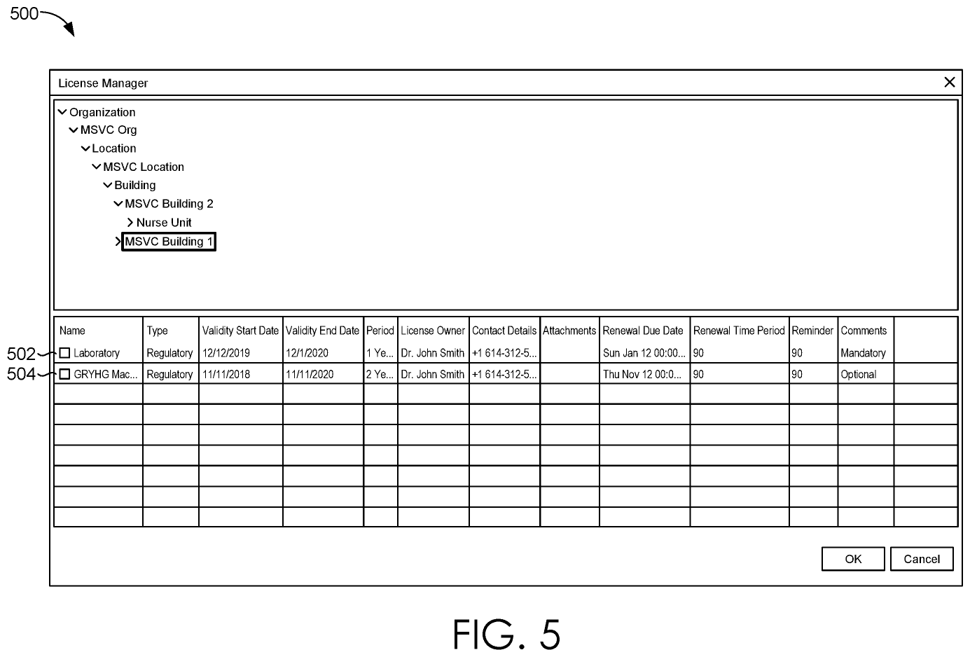

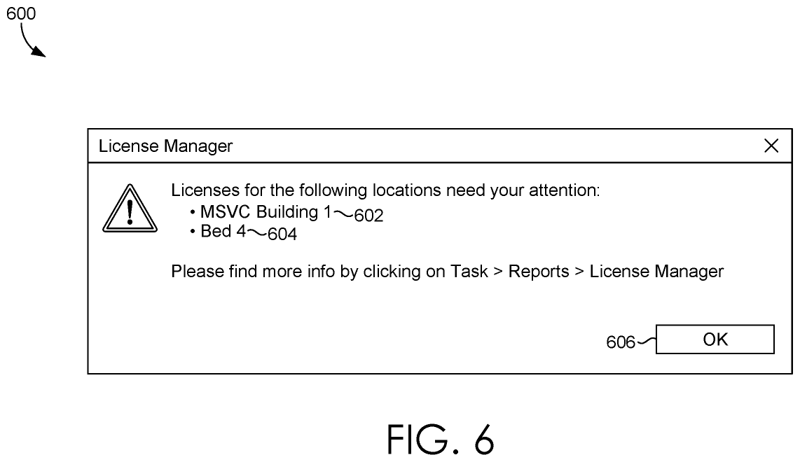

In embodiments, a dashboard owner 104 may use a secured system and organization security login 106 to gain access to the license manager tool 108. In embodiments, the license manager tool 108 may be associated with a database used to store information relevant to the licensed managed by the license manager tool 108. When the dashboard owner 104 can input one or more licenses which are stored in the database, whether local, remote, cloud-based, distributed, or a combination thereof, to ensure informational security due to the sensitive information in the licenses. The license manager tool 108 may be accessible through the dedicated graphical user interface associated with the intelligent license management application, which may be run locally, remotely, on a virtual server, in a distributed environment, or any combination thereof. Once the dashboard owner 104 has gained access to the license manager tool 108, for example using a login identifier and password, they may add additional licenses, update information associated with existing licenses, make note of expired licenses, or a combination thereof in accordance with their privilege level. Additionally, the dashboard owner 104 may use the intelligent license management application to set reminders for one or more particular licenses or upload newly-acquired or newly-renewed licenses. An exemplary embodiment of a graphical user interface 500 for the intelligent license management application and tool 108 will be discussed in association with FIG. 5 below, and an exemplary reminder notification 600 for displays in a graphical user interface will be discussed in association with FIG. 6 below.

FIG. 2 illustrates a license management and notification system 200 for intelligently managing licenses and generating notifications. The license management and notification system 200 may be stored in a database (now shown) associated with a healthcare facility 202. In further embodiments, the licenses management and notification system 200 may be associated with a healthcare facility 202. In embodiments, the healthcare facility 202 may be associated with an organization, entity, party, corporation, or the like that handles the administration of the healthcare facilities. The license management and notification system 200 may be associated with the organization. In such embodiments, the license management and notification system 200 associated with the organization may manage all the licenses 204 associated with the healthcare facility 202 associated with the organization. Although shown as singular, the healthcare facility 202 may correspond to any number of facilities, buildings, campuses, or the like.

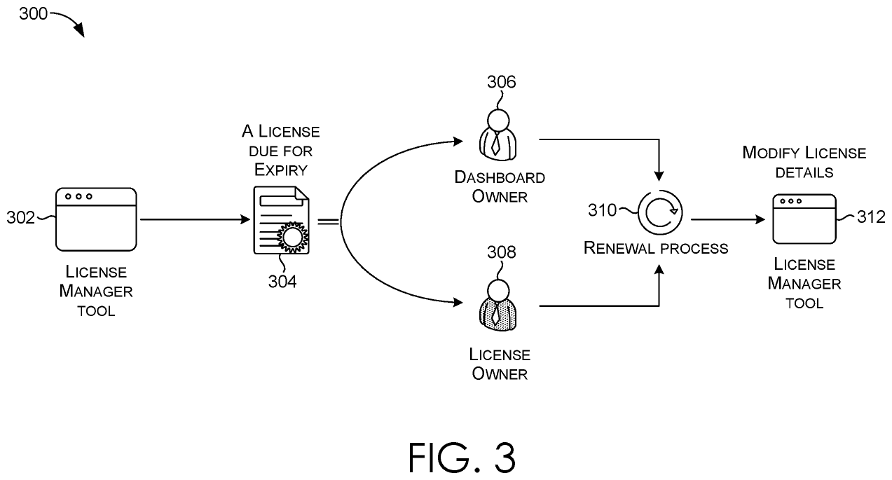

In embodiments, the dashboard owner 206 may use the add/modify license manager tool 208 of the license management and notification system 200 to add a new license or edit information associated with at least one existing license stored in association with the license management and notification system 200. The license management and notification system 200 may use this input information to encode renewal dates and complexity levels for the license(s) added or edited. As discussed in association with FIG. 3 below, the license management and notification system 200 may automatically determine, calculate, or convert this renewal date and complexity level into a reminder date using one or more processors, and further, may encode at least the renewal date, complexity level, and reminder date into a table associated with the license management and notification system 200. In embodiments, this renewal date may be used by the license management and notification system 200 to automatically generate a validity notification 210. The validity notification 210 may be transmitted by the license management and notification system 200 to a computing device associated with a user 212, such as personnel particular associated with the license, or other party designed for handling the license. In embodiments, the add/modify license manager tool 208 may identify and determine the correct computing device for routing the validity notification 210 based on contact information linked to the license associated with the reminder date, as stored in the database. The validity notification 210 may cause a graphical user interface to display, on the computing device associated with the user 212, a renewal date for a relevant license, a renewal complexity for the relevant license, information related to the relevant license, and/or a selectable link, for example.

In embodiments, the graphical user interface may receive a selection by the user 212. Based on receiving this selection, the add/modify license manager tool 208 may be caused to activate on the computing device associated with the user 212. In some embodiments, the add/modify license manager tool 208 may display information that is only related to the licenses to which the user is associated, such that the displayed information is limited to be relevant to the user 212 and/or limited to reflect a privilege level of the user 212. In various embodiments, the add/modify license manager tool 208 may display information related to the licenses associated with a healthcare facility 202 or organization, for example, based on an administrative privilege level associated with the user 212 to provide entity-wide or facility-wide visibility of licenses. The user 212 may optionally use the add/modify license manager tool 208 to provide an indication of an individual license renewed 214. For example, the user 212 may input information that specifies a physician's state license has been renewed for a particular year period (e.g., one year for state license in any discipline; ten years for board certification in neurology). In further embodiments, the graphical user interface may also be comprised of an upload link. The user 212 may use the upload link to provide the license management and notification system 200 with a copy of a renewed license. For example, a scanned image or PDF of a physical copy, or a virtual certificate with a token may be uploaded for storage in the database, to replace a corresponding existing and/or expired license in the database.

Having described license management and notification system 200 and components thereof, it will be understood by those of ordinary skill in the art that license management and notification system 200 is but one example of a suitable system and is not intended to limit the scope of use or functionality of the present disclosure. Similarly, license management and notification system 200 should not be interpreted as imputing any dependency and/or any requirements with regard to each component and combination(s) of components illustrated in FIG. 2. It will be appreciated by those of ordinary skill in the art that the location of components illustrated in FIG. 2 is an example, as other methods, hardware, software, components, and devices for establishing a communication links between the components shown in FIG. 2 may be utilized in implementations of the present disclosure. It will be understood to those of ordinary skill in the art that the components may be connected in various manners, hardwired or wireless, and may use intermediary components that have been omitted or not included in FIG. 2 for simplicity's sake. As such, the absence of components from FIG. 2 should not be interpreted as limiting the present disclosure to exclude additional components and combination(s) of components. Moreover, though components are represented in FIG. 2 as singular components, it will be appreciated that some embodiments may include a plurality of devices and/or components such that FIG. 2 should not be considered as limiting the number of a device or component.

FIG. 3 illustrates an exemplary flow chart 300 for intelligently generating notifications for licenses due for expiry. According to embodiments, a license manager tool 302 of the intelligent license management application, previously discussed, may use information stored in association with the application to determine that at least one license is a license due for expiry 304 (“due for expiry” refers to a predetermined date and/or time at which the license is no longer valid and has “expired” or “lapsed”). This may be determined based on information decoded by the license manager tool 302 from at least one license document stored in association with the intelligent license management application. A license document could be digital information associated with a license (as a data object that represents the license), or an electronic version (e.g., image or scan) of a physical document that has been downloaded and stored in association with the license. This information may then be converted into data regarding at least one of a renewal date, a complexity level, or contact information for a user associated with the license document. In embodiments, this converted data may be used by the intelligent license management application to identify, calculate, and/or determine reminder dates for the licenses stored in association with the intelligent license management application. As such, the intelligent license management application manages the reminder dates for each individual license in the database having tens to thousands of license, for example, independent of whether the license is specific to a facility, building, personnel, equipment, software, or combination thereof (e.g., a license to use specific software to control a particular device, such as a radiation beam device).

It then may be determined by the intelligent license management application that a renewal date corresponds with the current date. Based on this determination, the intelligent license management application may programmatically call the license manager tool 302 to transmit a notification to at least one of a dashboard owner 306 or a license owner 308. A license “owner” refers to an individual who is responsible for managing, overseeing, or handling license renewals to ensure continuity in proper and valid licensing for the license. In embodiments, the dashboard owner 306 may be associated with an administrative privilege level which indicates to the license manager tool 302 that each notification may be sent to the dashboard owner 306. In some embodiments, the license owner 308 may be associated with a standard privilege level. The standard privilege level may indicate to the license manager tool 302 that only notifications related to license(s) associated with the license owner 308 should be transmitted to the license owner 308. In various embodiments, the license owner 308 may be determined or identified based on information linked to the license due for expiry 304, and/or as stored in association with the database. For example, a name, physical and/or email address, telephone number, and/or other contact information may be linked to the license due for expiry 304 as stored in the table in the database. The contact information may be comprised of a telephone number, employee or personal email address, employee identification number, or any other information that may be used to contact the license owner. Based on receiving the transmitted notification, at least one of the dashboard owner 306 or license owner 308 may initiate, begin, undertake, and/or complete the renewal process 310. In embodiments, the notification may include specific and detailed information related to the process for renewing the application, such as details for when or how to renew the particular license. In further embodiments, the notification may include a selectable link which, when selected, may cause a website to display on a computing device associated with the dashboard owner 306 or license owner 308. As such, the selectable link may be generated by the intelligent license management application, wherein a website for the link has been stored in association with that particular license in the database. This website may have additional information related to the renewal process for the license, or may allow the dashboard owner 306 or license owner 308 to begin the renewal process 310 electronically through an external portal, for example. In embodiments, based on determining that the license due for expiry 304 has been renewed, the license manager tool 302 may automatically modify license details 312 to update the information associated with the license due for expiry 304, as stored in the database.

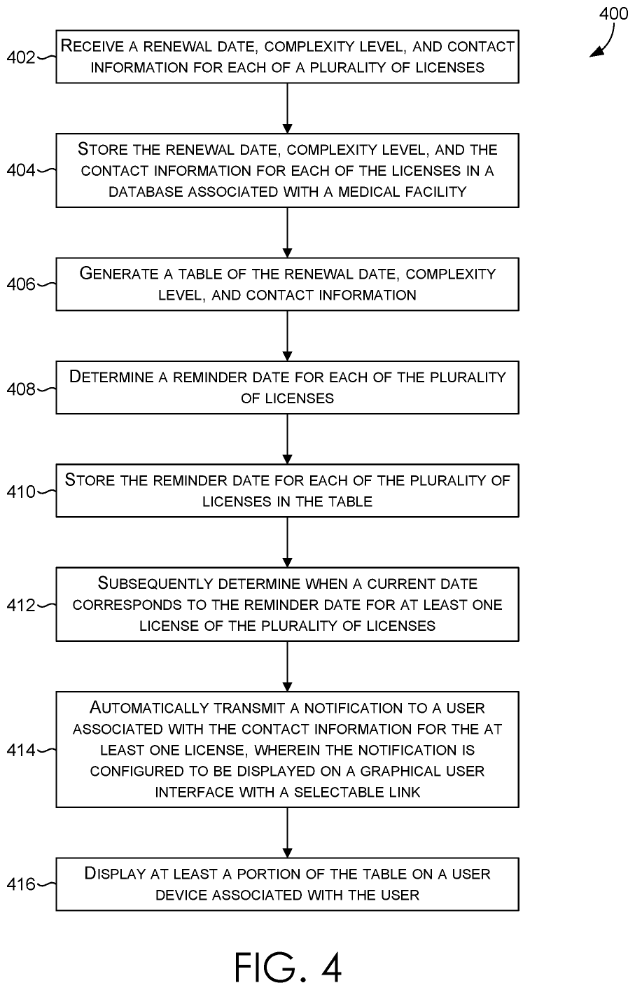

FIG. 4 illustrates a method 400 for intelligent license management using an intelligent license management user interface and application. The operations of the method 400 presented below are intended to be illustrative. In some implementations, the method 400 may be accomplished with one or more additional operations not described, or without one or more of the operations discussed. Additionally, the order in which the operations of the method 400 are illustrated in FIG. 4 and described below is not intended to be limiting unless expressly discussed.

Additionally, the method 400 of FIG. 4 may be performed via one or more of the components and component interactions of previously described FIGS. 1-3, and later described FIGS. 5-8. As such, the method 400 discussed briefly for brevity, though it will be understood that the previous discussion van be applicable to aspects of the method of FIG. 4. In various embodiments, one or more non-transitory computer-readable storage media having computer-readable instructions or computer-readable program code portions embodied thereon, for execution via one or more processors, may be executed to implement and/or perform one or more of the method 400 shown in FIG. 4. For example, computer-readable instructions or computer-readable program code portions can specify the performance of the method 400, can specify a sequence of steps of the method 400, and/or can identify particular component(s) of a software and/or hardware for performing one or more of the steps of the method 400, in embodiments. The computer-readable instructions or computer-readable program code portions can correspond to an application plugin or an application programming interface (API), in some embodiments. In one embodiment, the plugin or API can implement and/or perform the method 400. As discussed below, the method 400 can be performed using any and all of the software, hardware, modules, and/or component(s), shown in FIGS. 1-3, and 5-8.

In some implementations, method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium. The one or more processing devices may include one or more devices configured through hardware, firmware, and/or software to be specifically designed for execution of one or more of the operations of method 400.

FIG. 4 illustrates method 400, in accordance with one or more implementations. Method 400 may be implemented as a computer-implemented method. In further embodiments, method 400 may be implemented by a computer system having one or more computer storage media storing computer-useable instructions. In even further embodiments, method 400 may be implemented by non-transitory computer-readable media having computer-executable instructions embodied thereon that, when executed, perform a method.

In various embodiments, the method 400 for intelligent license management using an intelligent license management user interface and application may comprise, at block 402, receiving a renewal date, a complexity level, and contact information for each of a plurality of licenses in a database that is associated with a medical facility. Embodiments may also comprise receiving and storing an electronic copy of each license of the plurality of licenses in the database. In embodiments, the complexity level defines a total predicted quantity of days that are needed to process a renewal of the corresponding license, which can vary by the type of license and/or the regulatory entity that processes renewals for that particular license. As used herein, a complexity level may relate to an amount of time needed to renew a license. This amount of time may be determined based on a number of pages associated with a renewal application, an average delay associated with a renewal agency, or any other information related to time needed to renew a license. The complexity level may indicate that renewal of the corresponding license is a high complexity level, a medium complexity level, or a low complexity level, in various embodiments, to reflect ranges of renewal periods needed, wherein the complexity level increases with the total predicted quantity of days that are needed to process a renewal of the corresponding license. In some embodiments, when the corresponding license is associated with the high complexity level (due to the total predicted quantity of days that are needed to process the renewal of the corresponding license), the corresponding license is displayed in the graphical user interface using a first color. In such embodiments, when the corresponding license is associated with the medium complexity level, the corresponding license is displayed in the graphical user interface using a second color, and when the corresponding license is associated with the low complexity level, the corresponding license is displayed in the graphical user interface using a third color. The first, second, and third colors are different, generally, to provide a visual indicator and to visually distinguish between the complexity levels.

At block 404, the renewal date, complexity level, and the contact information for each of the plurality of licenses is stored in the database. As used herein, a medical facility and a healthcare facility describes a physical or digital location which is associated with at least one patient or information associated with at least one patient. The renewal date may describe a date by which a license must be renewed before the license lapses or is cancelled. The renewal date may also refer to a first date that a license is available to be renewed. In other aspects, the renewal date may refer to a date of expiration or expiry of the license on which the license ceases to operate to certify or validate its purpose. In embodiments, the contact information may refer to an email address, mail address, or phone number, as previously discussed, for contacting a user, license owner, license handled, or other entity. The contact information may be manually entered by a user, or may be determined based on information associated with a license.

At block 406, a table of the renewal date, the complexity level, and the contact information for each of the plurality of licenses is generated, wherein the table is stored in the database. The table may be generated based on decoding information related to a license and encoding said information into a format which may be organized, structured, and presented in a table albeit each license may be input, uploaded, downloaded, or otherwise obtained in various data formats and may include different types of information or data. The formatting of the table may be manually edited by a user, in some embodiments, using the application and tools mentioned herein. Additionally or alternatively, the formatting of the table may be automatically determined by the intelligent license management application to optimize the organization of the varied licenses.

At block 408, a reminder date for each of the plurality of licenses is determined based on the renewal date and the complexity level that corresponds to each of the plurality of licenses. In embodiments, the reminder date is determined by identifying, determining, and/or calculating a total number of days, actual, specifies, or predicted as needed to renew the license. The reminder date may also include the total number of days above, summed with an additional number of days in order to allow a “buffer” for a user to renew a license. In further embodiments, multiple renewal dates may be calculated to form a renewal “window” or a range of days/a period of time during which renewal can be managed. For example, it may be determined that a complexity level indicates that a time of six months is needed to renew a license, relative to the expiration date of the license. It may be determined that two reminder dates are needed for this renewal of the particular license, for example, a first reminder date may be determined to be one year prior to the renewal date, and a second reminder date may be determine to be six months prior to the renewal date. As such, each license may have one or more reminder dates to facilitate multiple notifications, as discussed below.

At block 410, the reminder date for each of the plurality of licenses is stored in the table, wherein the reminder date is stored in association with a corresponding license of the plurality of licenses. For each day of a calendar year, it is automatically determined by the application, for example, whether a current date corresponds to the reminder date for one or more of the plurality of licenses. At block 412, it is subsequently determined when a current date corresponds to the reminder date for at least one license of the plurality of licenses. For example, the intelligent license management application may determine automatically when a current date corresponds to a reminder date for any of the licenses in the database. At block 414, based on determining that the current date corresponds to the reminder date for the at least one license, a notification is automatically transmitted to a user associated with the contact information for the at least one license, wherein the notification is configured to be displayed on a graphical user interface with a selectable link. The intelligent license management application may cause one or more tools to trigger one or more notifications, as discussed prior. In further embodiments, the notification may be comprised of the renewal date, and the complexity level for the corresponding license. In embodiments, the user may be associated with either an administration privilege level or a standard privilege level. In further embodiments, the notification may cause intelligent license management application associated with the table to open on the computing device of the user, wherein the application causes the computing device to display the graphical user interface to the user.

At block 416, based on receiving an indication of a selection of the selectable link, at least a portion of the table is displayed on a user device associated with the user. In embodiments, the table (or portion thereof) is displayed based on determining whether the user is associated with the administration privilege level or the standard privilege level. In some embodiments, all contents of the table are displayed including the plurality of licenses on the user device associated with the user based on determining the user is associated with the administration privilege level. In other embodiments, only a portion of the table is displayed that is determined to include one or more of the plurality of licenses that are associated with the user, based on determining the user is associated with the standard privilege level, wherein another one or more of the plurality of licenses that are not associated with the user are omitted form the graphical user interface.

In embodiments, each license of the plurality of licenses having the renewal date closer (e.g., within a preset threshold number of days, such as 30 days or 14 business days) to the current date are displayed in the table at the topmost rows or “above” each license of the plurality of licenses having a renewal date farther form the current date (e.g., outside the preset threshold number of days), and each license having the renewal date farther from the current date are displayed below each license of the plurality of licenses having the renewal date closer to the current date. As such, the rows of the licenses in the table can be organized and presented in a ranked order such that those licenses having a nearest renewal date to the current date are located in the first/uppermost rows of the table, in descending order or renewal date. The at least a portion of the table displayed in the graphical user interface may also comprise a selectable option to upload a digital copy of the corresponding license. In further embodiments, a digital copy of the corresponding license is received based on receiving an indication of a selection of the selectable option. The renewal date for the corresponding license may then be updated based on receiving the digital copy of the corresponding license.

An additional embodiment comprises a non-transitory computer readable media having computer-executable instructions embodied thereon that, when executed, perform a method, the non-transitory computer readable media comprising a computer application plug-in. The computer application plug-in is configured for receiving a renewal date, a complexity level, and contact information for each of a plurality of license in a databased that is associated with a facility, wherein the complexity level defines a quantity of days that are needed to process a renewal of a corresponding license. Additionally, the renewal date defines a date for which the corresponding license is to expire, and the contact information defines a user identifier and a contact method that defines a user to be contacted as responsible for management of the corresponding license. In embodiments, a table of the renewal date, the complexity level, and the contact information for each of the plurality of licenses is automatically generated and stored by the intelligent license management application extracting information from the electronic data associated with the license.

For each of the one or more licenses in the plurality of licenses for which the current date corresponds to the reminder date, a notification is automatically transmitted to the user associated with the contact information for the one or more licenses, in further embodiments. The notification is configured to be displayed on a graphical user interface with a selectable link. Based on receiving an indication of a selection of the selectable link, at least a portion of the table is displayed on a computing device associated with the user. In further embodiments, a modification indicating that the corresponding license has been renewed and a digital copy of the corresponding license are received. The digital copy may also be stored in the database in association with the corresponding license that is renewed, for example, to overwrite an existing instance of the license and/or to update information for that license stored in the database. A new reminder date for the corresponding license may be calculated based on information associated with the digital copy, and the new reminder date for the corresponding license may be updated in the database.

Moving to FIG. 5, an exemplary graphical user interface 500 is illustrated in accordance with one or more implementations. In embodiments, the graphical user interface 500 may include all of the information displayed in FIG. 5, or it may include only portions of the information displayed in FIG. 5. For example, if a user is associated with a standard privilege level, the graphical user interface may only display the first row 502 and the second row 504. In further embodiments, the information displayed in the first row 502 may be associated with a first user, and the second row 504 may be associated with a second user. The notification discussed at least in association with FIG. 3 may cause only the first row 502 to display on a user device associated with the first user, and only the second row 504 on a user device associated with the second user. In embodiments, the intelligent license management application may cause display of all information in the table to a user associated with an administrative privilege level.

In embodiments, the first row 502 may store and/or display information related to a license which is due for renewal before the license associated with the information contained in the second row 504. Additionally, the information in the first row 502 may be displayed in a first color which differs from the second color of the second row 504. The differences in color may indicate a difference in complexity level (previously discussed), a difference in renewal or reminder date, or a difference in the type of license. In some embodiments, the type of license may refer to a mandatory versus optional license, or a medical license versus a license for different equipment associated with a facility or organization. In embodiments, the intelligent license management application may determine that the information stored in the first row 502 has a complexity level higher than the information stored in the second row 504. Based on this determination, the intelligent license management application may cause the first row 502 and the second row 504 to be displayed in different colors, as discussed prior. For example, the information in the first row 502 may be displayed in red and the information displayed in the second row 504 may be displayed in black. The red color may indicated that the license associated with the information in the first row 502 may have a higher complexity level than the license associated with the information in the second row 504.

In some embodiments, the intelligent license management application may determine which row in the table shown in a graphical user interface will display information associated with a particular license relative to one or more other licenses (displayed depending on privilege level). For example, the intelligent license management application may determine that a first license has a higher complexity level than a second license. Based on this determination, the intelligent license management application may cause information associated with the first license to be stored and/or displayed in the first row 502, and the information association with the second license to be stored and/or displayed in the second row 504, displayed below the first row. In other embodiments, the intelligent license management application may determine that a first license is associated with a renewal date that is closer to the current date than a renewal date associated with a second license. Based on this determination, the intelligent license management application may cause information associated with the first license to be displayed and/or stored in the first row 502, and information associated with the second license to be displayed and/or stored in the second row 504.

In another embodiment, the intelligent management application may determine that a first license is associated with a reminder date that is closer to the current date than a reminder date associated with a second license. Based on this determination, the intelligent license management application may cause information associated with the first license to be displayed and/or stored in the first row 502, and information associated with the second license to be displayed and/or stored in the second row 504. In various embodiments, the intelligent management application may also generate and a display a table which organizes and/or stores information associated with a single healthcare facility or entity. Additionally, the intelligent management application may generate and display a table which organizes and/or stores information associated with each healthcare facility or entity that is associated with an organization. The intelligent management application may generate and display a table organizing information associated with any number and kind/type of licenses associated with any number of organizations, healthcare facilities, entities, or any combination thereof.

Moving to FIG. 6, an exemplary license management notification 600 is illustrated in accordance with one or more implementations. In embodiments, the license management notification 600 may display information associated with a license. In this example license management notification 600, the building of a healthcare facility is displayed in the first information section 602, and the equipment is displayed in the second information section 604. In embodiments, any information related to a license associated with the notification may be displayed within the license management notification 600, the information illustrated at the first information section 602 and the second information section 604 are just examples.

Additionally, the license management notification 600 may include a selectable link 606. In embodiments, the intelligent license management application may determine that a notification has been received by a user based on detecting an selection of the selectable link 606. In embodiments, the selectable link 606 may comprise various other functions and the examples herein are not limiting. For example, the selectable link 606 may link the notification directly to the intelligent license manager application. Based on receiving a selection of the selectable link 606, the intelligent license manager application may be caused to activate on the user's computing device. This activation may cause the display of at least a portion of the table generated by the intelligent license management application. The selectable link 606 be configured to cause display of an option to upload a digital copy of a license or a renewal certificate for a license. Upon receiving the digital copy of the license or renewal certificate for the license, the intelligent license management application may determine at least a new renewal date or a new renewal complexity for the license. The intelligent license management system may then update the information stored and/or displayed in the table based on the uploaded digital copy of the license or renewal certificate for the license.

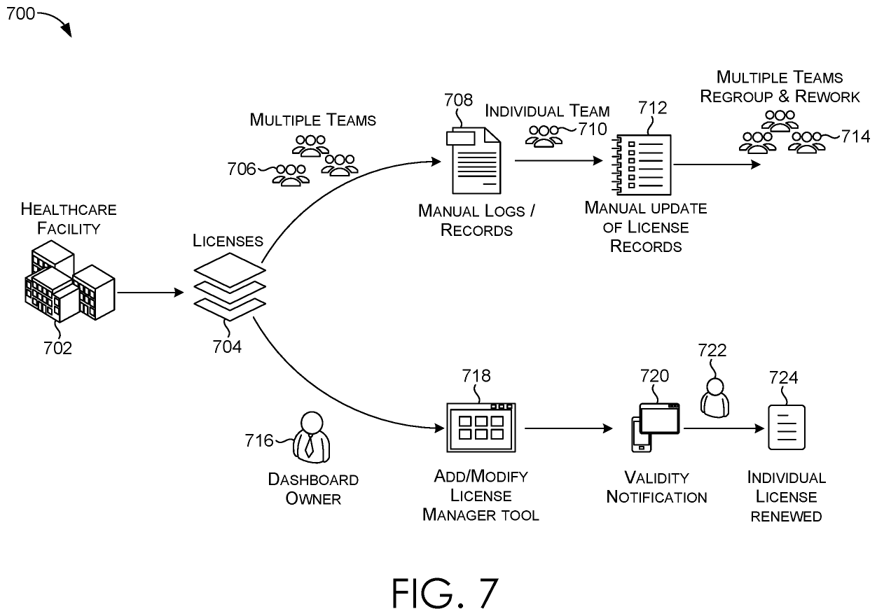

Moving to FIG. 7, an exemplary flow chart 700 for intelligently generating license management notifications for teams. In embodiments, a healthcare facility 702 may be associated with a plurality of licenses 704. At least one of the licenses 704 may be associated with multiple teams 706 and/or a dashboard owner 716. The flow chart 700 illustrates that multiple teams may use the intelligent license management application to view and/or edit the manual logs/records 708 associated at least one of the multiple teams 706. The individual team 710 associated with at least one of the licenses 704 associated with the healthcare facility 702 may conduct a manual update of license records 712 associated with the individual team 710. In embodiments, at least one member of the individual team 710 may be able to use the intelligent management application to regroup and rework multiple teams 714. For example, a set of licenses may be associated with a maintenance team. If a license associated with the maintenance team has a reminder date proximate to a determined current date, the intelligent license management application may generate and transmit notifications for each member of the maintenance team, to multiple different devices for those team members/users. The intelligent license management application may also store a contact individual of the individual team members/users. In embodiments, the intelligent management application may transmit a renewal notification to only one contact individual.

At least one user associated with an individual team 710 may also be associated with an administrative privilege level. The administrative privilege level may indicate to the intelligent license management application that the user may regroup and rework existing teams, or create new teams. For example, a user associated with an administrative privilege level may manually associated themselves or other users with any number of teams. Additionally a user associated with an administrative privilege level may disassociate users associated with an individual team 710 and associate them with a different individual team 710.

In embodiments, a dashboard owner 716 may also use an add/modify license manager tool 718 of the intelligent license management application to add or modify licenses associated with at least one individual team 710. The dashboard owner 716 may also manually configure notification preferences to determine which user 722 associated with which team will receive a validity notification 720. Similar to other embodiments, the user 722 notified, or any other user associated with a team may renew a license and provide an indication of an individual license renewed 724.

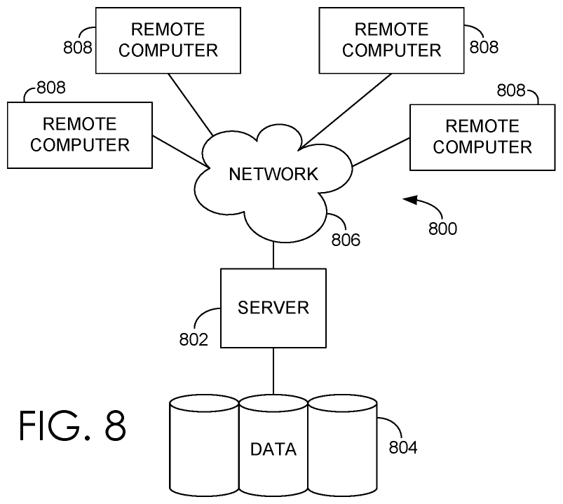

Hereinafter, an example computing environment is described with regard to the systems, methods, and computer-media described hereinabove. Turning to FIG. 8, an example computing environment is depicted, in accordance with an embodiment of the present disclosure. It will be understood by those of ordinary skill in the art that the example computing environment 800 is just one example of a suitable computing environment and is not intended to limit the scope of use or functionality of the present disclosure. Similarly, the computing environment 800 should not be interpreted as imputing any dependency and/or any requirements with regard to each component and combination(s) of components illustrated in FIG. 8. It will be appreciated by those having ordinary skill in the art that the connections illustrated in FIG. 8 are also examples as other methods, hardware, software, and devices for establishing a communications link between the components, devices, systems, and entities, as shown in FIG. 8, may be utilized in implementation of the present disclosure. Although the connections are depicted using one or more solid lines, it will be understood by those having ordinary skill in the art that the example connections of FIG. 8 may be hardwired or wireless, and may use intermediary components that have been omitted or not included in FIG. 8 for simplicity's sake. As such, the absence of components from FIG. 8 should be not be interpreted as limiting the present disclosure to exclude additional components and combination(s) of components. Moreover, though devices and components are represented in FIG. 8 as singular devices and components, it will be appreciated that some embodiments may include a plurality of the devices and components such that FIG. 8 should not be considered as limiting the number of a device or component.

Continuing, the computing environment 800 of FIG. 8 is illustrated as being a distributed environment where components and devices may be remote from one another and may perform separate tasks. The components and devices may communicate with one another and may be linked to each other using a network 806. The network 806 may include wireless and/or physical (e.g., hardwired) connections. Examples of networks include a telecommunications network of a service provider or carrier, Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. The network 806, generally, provides the components and devices access to the Internet and web-based applications.

The computing environment 800 comprises a remote computer 808, which may be in the form of a server 802, as shown in the example of FIG. 8. Although illustrated as one component in FIG. 8, the present disclosure may utilize a plurality of local servers and/or remote servers in the computing environment 800. The remote computer 808 may include components such as a processing unit, internal system memory, and a suitable system bus for coupling to various components, including electronic storage, memory, and the like, such as a data store, a database, and/or a database cluster. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced Industry Standard Architecture (EISA) bus, Video Electronics Standards Association (VESA®) local bus, and Peripheral Component Interconnect (PCI) bus, also known as Mezzanine bus.

The remote computer 808 may include or may have access to computer-readable media. Computer-readable media can be any available media that may be accessed by remote computer 808, and includes volatile and nonvolatile media, as well as removable and non-removable media. By way of example, and not limitation, computer-readable media may include computer storage media and communication media. Computer storage media may include, without limitation, volatile and nonvolatile media, as well as removable and non-removable media, implemented in any method or technology for storage of information, such as computer-readable instructions, data structures, program modules, or other data. In this regard, computer storage media may include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the remote computer 808. Computer storage media does not comprise signals per se.

Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above also may be included within the scope of computer-readable media.

In embodiments, the remote computer 808 uses logical connections to communicate with one or more remote computers 808 within the computing environment 800. In embodiments where the network 806 includes a wireless network, the remote computer 808 may employ a modem to establish communications with the Internet, the remote computer 808 may connect to the Internet using Wi-Fi or wireless access points, or the server 802 may use a wireless network adapter to access the Internet. The remote computer 808 engages in two-way communication with any or all of the components and devices illustrated in FIG. 8, using the network 806. Accordingly, the remote computer 808 may send data to and receive data from the remote computers 808 over the network 806.

Although illustrated as a single device, the remote computers 808 may include multiple remote computers. In an embodiment having a distributed network, the remote computers 808 may be located at one or more different geographic locations. In an embodiment where the remote computers 808 is a plurality of remote computers, each of the plurality of remote computers may be located across various locations such as buildings in a campus, medical and research facilities at a medical complex, offices or “branches” of a banking/credit entity, or may be mobile devices that are wearable or carried by personnel, or attached to vehicles or trackable items in a warehouse, for example.

In some embodiments, the remote computers 808 are physically located in a medical setting such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. By way of example, a medical professional may include physicians; medical specialists such as surgeons, radiologists, cardiologists, and oncologists; emergency medical technicians; physicians' assistants; nurse practitioners; nurses; nurses' aides; pharmacists; dieticians; microbiologists; laboratory experts; genetic counselors; researchers; veterinarians; students; and the like. In other embodiments, the remote computers 808 may be physically located in a non-medical setting, such as a packing and shipping facility or deployed within a fleet of delivery or courier vehicles.

Continuing, the computing environment 800 includes a data store 804. Although shown as a single component, the data store 804 may be implemented using multiple data stores that are communicatively coupled to one another, independent of the geographic or physical location of a memory device. Examples of data stores may store data in the form of artifacts, server lists, properties associated with servers, environments, properties associated with environments, computer instructions encoded in multiple different computer programming languages, deployment scripts, applications, properties associated with applications, release packages, version information for release packages, build levels associated with applications, identifiers for applications, identifiers for release packages, users, roles associated with users, permissions associated with roles, workflows and steps in the workflows, clients, servers associated with clients, attributes associated with properties, audit information, and/or audit trails for workflows. Examples of data stores may also store data in the form of electronic records, for example, electronic health records of patients, transaction records, billing records, task and workflow records, chronological event records, and the like.

Generally, the data store 804 includes physical memory that is configured to store information encoded in data. For example, the data store 804 may provide storage for computer-readable instructions, computer-executable instructions, data structures, data arrays, computer programs, applications, and other data that supports the functions and action to be undertaken using the computing environment 800 and components shown in the example of FIG. 8.

In a computing environment having distributed components that are communicatively coupled via the network 806, program modules may be located in local and/or remote computer storage media including, for example only, memory storage devices. Embodiments of the present disclosure may be described in the context of computer-executable instructions, such as program modules, being executed by a computing device. Program modules may include, but are not limited to, routines, programs, objects, components, and data structures that perform particular tasks or implement particular abstract data types. In embodiments, the remote computer 808 may access, retrieve, communicate, receive, and update information stored in the data store 804, including program modules. Accordingly, the remote computer 808 may execute, using a processor, computer instructions stored in the data store 804 in order to perform embodiments described herein.

Although internal components of the devices in FIG. 8, such as the remote computer 808, are not illustrated, those of ordinary skill in the art will appreciate that internal components and their interconnection are present in the devices of FIG. 8. Accordingly, additional details concerning the internal construction device are not further disclosed herein.

Also, the present has been described in relation to particular embodiments, which are intended in all respects to be illustrative rather than restrictive. Thus the present disclosure is not limited to these embodiments, but variations and modifications may be made without departing from the scope of the present disclosure.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A method comprising:
receiving a renewal date, a complexity level, and contact information for each of a plurality of licenses in a database that is associated with a medical facility;
storing at least the renewal date, the complexity level, and the contact information for each of the plurality of licenses in the database;
generating a table of the renewal date, the complexity level, and the contact information for each of the plurality of licenses, wherein the table is stored in the database;
determining a reminder date for each of the plurality of licenses based on the renewal date and the complexity level that corresponds to each of the plurality of licenses;
storing the reminder date for each of the plurality of licenses in the table, wherein the reminder date is stored in association with a corresponding license of the plurality of licenses;
subsequently determining when a current date corresponds to the reminder date for at least one license of the plurality of licenses;
based on determining that the current date corresponds to the reminder date for the at least one license, automatically transmitting a notification to a user associated with the contact information for the at least one license, wherein the notification is configured to be displayed on a graphical user interface with a selectable link; and
based on receiving an indication of a selection of the selectable link, displaying at least a portion of the table on a user device associated with the user.

2. The method of claim 1, wherein the complexity level defines a quantity of days that are needed to process a renewal of the corresponding license.

3. The method of claim 1, wherein the complexity level indicates that renewal of the corresponding license is a high complexity level, a medium complexity level, or a low complexity level.

4. The method of claim 3, wherein when the corresponding license is associated with the high complexity level, the corresponding license is displayed in the graphical user interface using a first color, wherein when the corresponding license is associated with the medium complexity level, the corresponding license is displayed in the graphical user interface using a second color, and wherein when the corresponding license is associated with the low complexity level, the corresponding license is displayed in the graphical user interface using a third color.

5. The method of claim 1, wherein the user is associated with either an administration privilege level or a standard privilege level.

6. The method of claim 5, wherein displaying the table further comprises:
determining whether the user is associated with the administration privilege level or the standard privilege level.

7. The method of claim 6, wherein displaying the table further comprises:
based on determining the user is associated with the administration privilege level, displaying all contents of the table, including the plurality of licenses, on the user device associated with the user.

8. The method of claim 6, wherein displaying the table further comprises:
based on determining the user is associated with the standard privilege level, displaying only the portion of the table that is determined to include one or more of the plurality of licenses that are associated with the user, wherein another one or more of the plurality of licenses that are not associated with the user are omitted from the graphical user interface.

9. The method of claim 1, further comprising:
displaying in the table each of the plurality of licenses having the renewal date closer to the current date above each of the plurality of licenses having the renewal date farther from the current date, and displaying each of the plurality of licenses having the renewal date farther from the current date below each of the plurality of licenses having the renewal date closer to the current date.

10. The method of claim 1, further comprising receiving and storing an electronic copy of each license of the plurality of licenses in the database.

11. Non-transitory computer readable media having computer-executable instructions embodied thereon that, when executed, perform a method, the non-transitory computer readable media comprising:
a computer application plug-in configured for:
receiving a renewal date, a complexity level, and contact information for each of a plurality of licenses in a database that is associated with a facility, wherein the complexity level defines a quantity of days that are needed to process a renewal of a corresponding license, wherein the renewal date defines a date for which the corresponding license is to expire, and wherein the contact information defines a user identifier and a contact method that defines a user to be contacted as responsible for management of the corresponding license;
automatically generating and storing a table of the renewal date, the complexity level, and the contact information for each of the plurality of licenses, wherein the table is stored in the database that is associated with a particular facility, and wherein the plurality of licenses correspond to one or more licenses that govern personnel or equipment at the particular facility;
automatically calculating a reminder date for each of the plurality of licenses based on the renewal date and the complexity level that corresponds to each of the plurality of licenses;
automatically storing the reminder date for each of the plurality of licenses in the table, wherein the reminder date is stored in association with the corresponding license in the plurality of licenses;
for each day of a calendar year, automatically determining whether a current date corresponds to the reminder date for one or more of the plurality of licenses;
for each of the one or more licenses in the plurality of licenses for which the current date corresponds to the reminder date, automatically transmitting a notification to the user associated with the contact information for the one or more licenses, wherein the notification is configured to be displayed on a graphical user interface with a selectable link; and
based on receiving an indication of a selection of the selectable link, displaying at least a portion of the table on a user device associated with the user.

12. The non-transitory computer readable media of claim 11, further comprising receiving a modification that indicates the corresponding license is renewed, and receiving a digital copy of the corresponding license.

13. The non-transitory computer readable media of claim 12, further comprising storing the digital copy, in association with the corresponding license, in the database.

14. The non-transitory computer readable media of claim 13, further comprising:
calculating a new reminder date for the corresponding license based on information associated with the digital copy; and
updating the reminder date for the corresponding license in the database.

15. A computer system comprising:
a memory;
one or more processors; and
one or more computer storage media storing computer-usable instructions that, when used by the one or more processors, cause the one or more processors to:
receive a renewal date, a complexity level, and contact information for each of a plurality of licenses in a database that is associated with a medical facility;
store at least the renewal date, the complexity level, and the contact information for each license of the plurality of licenses in the database;
generate a table of the renewal date, the complexity level, and the contact information for each of the plurality of licenses, wherein the table is stored in the database;
determine a reminder date for each of the plurality of licenses based on the renewal date and the complexity level that corresponds to each of the plurality of licenses;
store the reminder date for each of the plurality of licenses in the table, wherein the reminder date is stored in association with a corresponding license of the plurality of licenses;
subsequently determine when a current date corresponds to the reminder date for at least one license of the plurality of licenses;
based on determining that the current date corresponds to the reminder date for the at least one license, automatically transmit a notification to a user associated with the contact information for the at least one license, wherein the notification is configured to be displayed on a graphical user interface of a user device with a selectable link; and
based on receiving an indication of a selection of the selectable link, display at least a portion of the table on the user device associated with the user.

16. The computer system of claim 15, wherein the notification causes an application associated with the table to open on the user device, wherein the application causes the user device to display the graphical user interface.

17. The computer system of claim 15, wherein the notification further comprises the renewal date, and the complexity level for the corresponding license.

18. The computer system of claim 15, wherein the at least a portion of the table comprises a selectable option to upload a digital copy of the corresponding license.

19. The computer system of claim 18, wherein the one or more processors are configured to:
based on receiving an indication of a selection of the selectable option, receive the digital copy of the corresponding license.

20. The computer system of claim 19, wherein the one or more processors are configured to:
updating the renewal date for the corresponding license based on receiving the digital copy of the corresponding license.
